(19) United States
(12) Patent Application Publication
(10) Pub. No.: US 2025/0009387 A1
(43) Pub. Date: Jan. 9, 2025

(54) APPARATUS WITH STYLET FOR KINK NAVIGATION OR THE LIKE IN A CATHETER LEAD LUMEN

(71) Applicant: KONINKLIJKE PHILIPS N.V., EINDHOVEN (NL)

(72) Inventor: AARON DOMINGO LEYVA, AURORA, CO (US)

(21) Appl. No.: 18/711,636

(22) PCT Filed: Nov. 14, 2022
(86) PCT No.: PCT/EP2022/081693

§ 371 Date: May 20, 2024

Related U.S. Application Data
(60) Provisional application No. 63/282,066, filed on Nov. 22, 2021.

(51) Int. Cl. A61B 17/34; A61M 25/00

(52) U.S. Cl. CPC A61B 17/3468; A61M 25/0043; A61M 2025/0019; A61M 2025/0063

(57) ABSTRACT

Apparatuses for lead extraction using a lead locking device are provided. The apparatus includes a stylet device configured to insert into a pathway of a lumen of the catheter including the first part of a tip; and the second part of a body wherein the body is configured at an end for connection to the tip when deployed in the lumen of the catheter; wherein the tip of the stylet device is configured to: apply pressure at an obstruction in the catheter, remove the obstruction by the movement against the obstruction to cause a clearing of obstruction in the lumen of the catheter, and enable the opening of the catheter for a lead locking device (LLD) to navigate past during the lead extraction procedure.

### TECHNICAL FIELD

The technical field generally relates to endovascular procedures and more particularly relates to apparatuses for assisting in endoscopic lead extraction by using a stylet configured with a tip profile that includes an off-centered tip angle and is connected to an electromechanical handle to apply movement to the stylet while the stylet is inserted in a catheter of lead to alleviate kinks in the catheter to navigate a lead locking device (LLD) in the catheter during a lead extraction procedure.

### BACKGROUND

In general, a current lead extraction procedure is performed by a medical provider with the insertion of a lead locking device (LLD) down the lead lumen and pulling on the lead statically to determine if the lead is removable. In this procedure, the clearing or pathway in the lead lumen can be obstructed resulting in difficulties by the medical provider to insert fully the LLD the length of the lead lumen. Often the cause of the obstruction is kinks or bends that have occurred by twisting and movement of the lead catheter, and also by scaring of about the leads; the result is that the extraction procedure can be delayed as the medical provide attempts to navigate the kinks or bend, or may have to perform the extraction procedure with the LLD not traversing the entire length of the lead lumen because of the difficulties to navigate down the lead lumen. The pathway is required or needed to be clear so that the lead locking device can be inserted far enough into the lead (i.e., the length of the lead is preferred) for the lead to be properly deployed, and for a higher likelihood of successful extraction. Further, without the proper deployment of the lead to the entire length of the lead lumen, it can be medically deemed detrimental to enabling a successful removal of the lead in the extraction part of the procedure and may give a medical provider pause to cease the extraction procedure.

It is desirable to implement a lead navigation device and system of a stylet device configured with a flexible tip that can be inserted endoscopically to clear the kink or the bend in the catheter during the lead extraction procedure.

It is desirable to implement to the stylet device for lead navigation, an ability to apply movement in the catheter to overcome resistance caused by the kink or bend of the catheter to relax the catheter and open an obscured or blocked lumen of the catheter for the convenient insertion of the LLD during the lead extraction procedure.

Accordingly, technologically improved systems and methods for valve resection and reshaping using a catheter with a resection tool to perform the is desirable. The following disclosure provides these technological enhancements, in addition to addressing related issues.

### BRIEF SUMMARY

This summary is provided to describe select concepts in a simplified form that are further described in the Detailed Description. This summary is not intended to identify key or essential features of the claimed subject matter, nor is it intended to be used as an aid in determining the scope of the claimed subject matter.

In an embodiment, an apparatus to assist in a lead extraction procedure of a catheter is provided. The apparatus includes a stylet device configured to insert into a pathway of a lumen of the catheter including the first part of a tip; and the second part of a body wherein the body is configured at an end for connection to the tip when deployed in the lumen of the catheter; wherein the tip of the stylet device is configured to apply pressure at an obstruction in the catheter: remove the obstruction by movement against the obstruction to cause a clearing of the obstruction in the catheter, and enable the opening of the lumen of the catheter for a lead locking device (LLD) to navigate past during the lead extraction procedure.

Furthermore, other desirable features and characteristics of the system and method will become apparent from the subsequent detailed description and the appended claims, taken in conjunction with the accompanying drawings and the preceding background.

### BRIEF DESCRIPTION OF THE DRAWINGS

The present application will hereinafter be described in conjunction with the following drawing figures, wherein like numerals denote like elements, and:

FIG. 1 illustrates an exemplary diagram of a structure of the non-invasive tool for lead extraction implementing a hammer drill and torque device, in which the extraction tool is not required to penetrate the body cavity during the lead extraction procedure to assist in the lead extraction procedure in accordance with an embodiment;

FIG. 2 illustrates an exemplary diagram of an alternative embodiment of replacing the hammer drill with a vibration source of the lead extraction assistance device in accordance with an embodiment; and

FIG. 3 illustrates is an exemplary diagram of implementing a combination of the hammer drill and a vibration source described in FIGS. 1 and 2 in a single assembly in the lead extraction assistance device in accordance with an embodiment.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Thus, any embodiment described herein as “exemplary” is not necessarily to be construed as preferred or advantageous over other embodiments. The embodiments described herein are exemplary embodiments provided to enable persons skilled in the art to make or use the invention and not to limit the scope of the invention that is defined by the claims. Furthermore, there is no intention to be bound by any expressed or implied theory presented in the preceding technical field, background, summary, or the following detailed description.

With increased numbers of cardiac implantable electronic devices (CIEDs) such as pacemakers, defibrillators (ICD), and cardiac resynchronization therapy (CRT) devices in patients, and consistent with the higher numbers of CIED implanted, there is also more complications, infections, and malfunctions by the greater number of CIEDs in use that necessitate more lead extraction procedures. A rationale for lead removal is given for both infectious and non-infectious conditions: the non-infectious condition can include malfunctioning leads or leads which can cause harm to the patient while the presence of an infection is for infection-based lead removal.

Embodiments provide a technical solution to this problem in the form of a lead navigation system that enables deploying a stylet tool configured with a flexible tip that addresses an issue with lead removal during the preparation of an extraction procedure. Specifically, the lead catheter can develop kinks or bends in rings that cause blockages in the pathway of the lead catheter. In this instance, the inner lumen of the catheter is compromised by bends or kinks in its path and the compromised catheter is difficult to clear manually by threading of the locking (or clearing) stylet, leading a medical provider to consider abandoning lead and a failed attempt at extraction.

In embodiments, the present disclosure describes a lead navigation system of a stylet device that is made up of a mandrel (of specified diameter) that has a half-tapered tip profile at an extreme angle and long length that allows its tip to make entry into a sharp kink. Once in the kink, the motorized portion will rotate and reciprocate (in and out when looking through) to overcome the force of the kink, eventually bending it back to its original shape and clearing the path allowing correct positioning of a lead locking device during deployment. This results in an effective lead removal by the preparation of the lead position throughout the length of the lead lumen to allow for a strong locking interface between the outer portions of the lead locking device with the inner portion of the lead (i.e., lead lumen interior). With an ability to overcome obstacles in threading the LLD such as kinks and bends obscuring a clear pathway of the lead catheter, it is foreseen that a reduction in the number of lead abandonment cases will entail that are caused due to the inability to effectively lock onto cardiac pacemaker leads.

The figures and descriptions below provide more detail.

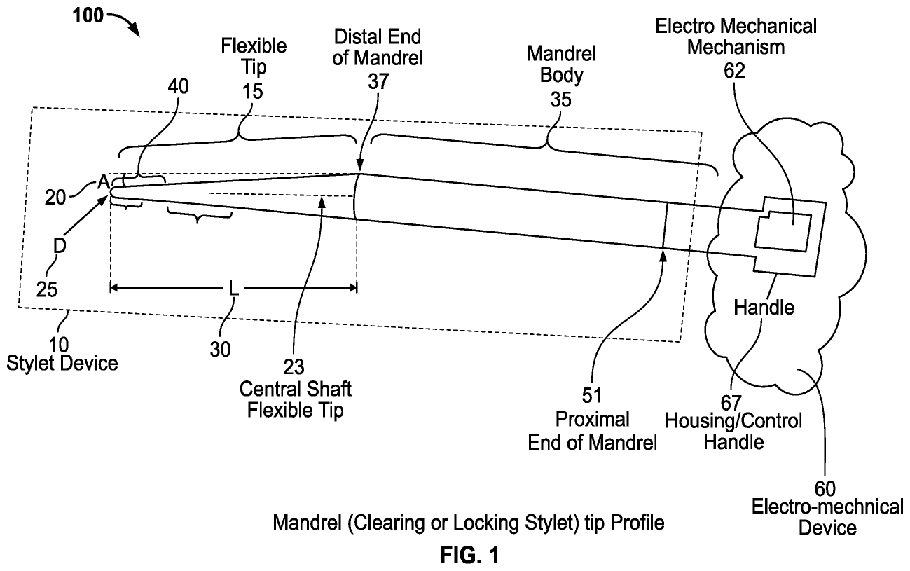

Turning now to FIG. 1 in an embodiment, FIG. 1 illustrates a diagram of a lead navigation system of the stylet device with the flexible tip to assist preparation of lead extraction, and in an embodiment with the use of a lead locking device (LLD) during a lead extraction procedure.

In an embodiment, the lead navigation system 100 assists in clearing a pathway in a vessel for the guidance of a guidewire or other obstructions. Hence, while the below disclosure describes the implementation of the lead navigation system 100 for lead extraction procedures, it is contemplated that the lead navigation system 100 is applicable for the guidance of other instruments in endoscopic procedures within a lumen of a catheter or like device, as well as directly endoscopic instruments that may encounter obstructions in lumens of arteries and other vessels in a body cavity.

In an embodiment, the lead navigation system 100 may be used to clear a pathway to assist, as an example, a guidewire in an endoscopic procedure, or in a related procedure in combination with the lead extraction procedure.

In FIG. 1 in the system 100 for lead navigation, there is shown a stylet device 10 that includes a flexible tip 15 and mandrel body 35 that is connected to the flexible tip 15 at the distal end 37 of the mandrel body 35. The proximal end 51 of the mandrel body is coupled to the electromechanical device 60.

In an embodiment, the lead navigation system 100 is composed of a mandrel body 35 with a specific diameter 25, and tip profile of the flexible tip 15, and an electromechanical device 60 that provides a rotational and reciprocating mechanism for the mandrel body 35. The tip profile includes a tip diameter 25, tip angle 20, and tip length 30 all found at the distal end 39 of the mandrel body 35. The proximal end 51 of the mandrel body 35 that makes up the stylet device 10 connects the stylet device 10 to the control handle 67 of the electromechanical device 60 and provides electromechanical control operations of the twist-action and push-pull motions to the stylet device 10. The tip includes a radiopaque tip 40 for identification/tracking under fluoroscopy. A marking 45 on the outside of the device to identify the rotational position in reference to the tip features.

During a lead extraction procedure, a lead extracting medical provider removes the tip of the lead to expose a catheter of lead and to allow for insertion of for extraction a Lead Locking Device (LLD). In an embodiment, the SPECTRANETICS® Lead Locking Device is used in the lead extraction procedure, for threading through the lead lumen past the formed obstruction once cleared at it provide a stable traction platform by locking along the entire contacted lead lumen (i.e., along the entire length past any kinks, bends, etc.).

In embodiments, the medical provider may initiate the lead extraction process by undressing each of the leads which include removing the insulation about the lead catheter and decoupling the outer coil carefully to gain access to the inner coil. Once the inner coil of the lead catheter is exposed and expanding stylet is inserted to expand the inner coil of a lead catheter in preparation for insertion of the clearing stylet (of appropriate size for the identified lead catheter), that is snaked down the lead lumen to clear the path of the inner coil for later insertion of the LLD. In instances, during this process, the medical provider may have difficulty inserting the clearing stylet and/or the LLD because of kinks, bends, or other obstructions formed in the lead catheter. In this case, the medical provider may elect to use the lead navigation system 100 with the stylet device 10 and an electromechanical device 60 that provides a rotational and reciprocating mechanism for the mandrel body 35 to remove a detected kink, bend, or other obstruction, and then reinsert the clearing stylet or LLD in the lead extraction procedure.

In an embodiment, the stylet device 10 is configured to insert into a pathway the approximate length of the entire lead lumen of the lead catheter to clear any obstruction located at any point in the pathway of the lead lumen. The mandrel body is configured at a distal end 39 for connection to the flexible tip 15 and both the mandrel body 35 and flexible tip 15 are deployed endoscopically in the lumen of the lead catheter. The stylet device 10 is inserted into the pathway of the lead lumen to a location of the situs of an obstruction formed in the lead catheter. The obstruction may be deemed to be a kink in the catheter, a bend, or other malformation that causes the pathway to be obscured or blocked, and to prevent the LLD from being inserted past the blockage. The stylet device 10 is configured by the connection of the mandrel body 35 to the flexible tip 15 to receive pressure, vibrations, or other forces by motions that include twisting and back/forth movement applied by the electromechanical mechanism 62 that is attached to the stylet device 10 that can either in an automated manner or my manual hand movement by the medical provider provide pressure from twisting and/or back/forth actions (i.e., manipulations) to the situs to of the formed obstruction in the lead catheter. The kinks and bends the manipulations of the stylet device 10 that are translated to the flexible tip 15, can cause the flexible tip 15 to press against the formed obstruction in the lead catheter to dislodge, release, and/or loosen the obstruction to clear the pathway.

In embodiments, the flexible tip 15 is constructed of a material that allows for a certain amount of flexibility when pressed against a kink or bend in the lead catheter to prevent damage or perforating of the catheter if an inordinate amount of pressure is applied to overcome the kink or bend resistance.

In an embodiment, the flexible tip 15 can exert pressure against an inner lumen side of the lead lumen to overcome the resistance of the lead catheter at a point of kink or bend and unflex or unkink the lead catheter back to an original position or to a position that opens the pathway sufficient for the clearing stylet or LLD to pass during the lead extraction procedure.

In an embodiment, the flexible tip 15 of the stylet device 10 is rounded at the distal end 22 to prevent damage or perforation to the lead lumen when pressed against the formed obstruction in response to a rotation and the back/forth motion. The flexible tip 15 is configured in accordance with a flexible tip profile in which the flexible tip profile includes constraints or attributes of a tip length, a tip diameter, and a taper angle.

The tip length 30 of the flexible tip 15 profile is half-tapered in a longitudinal direction to enable the flexible tip to penetrate the kink of the lead catheter, and this is especially useful in the case of a kink which is sharply angled causing an almost complete closure of the pathway of the lead lumen. In this instance, the flexibility and the tapering of the flexible tip 15 allow a medical provider to thread it into the kink or bend, and to exert pressure to unflex the kink or bend back to an original position.

The tip diameter 25 is sized in accordance with a coil profile of the lead catheter to enable the flexible tip 15 to maneuver about coils of the lead catheter and to prevent lodgement in a coil of the lead catheter. In embodiments, the mandrel body (which the flexible tip is coupled to) is sized in accordance with an LLD which has a diameter of a range of approximately 0.013″/0.33 mm to 0.016″/0.41 mm, and 0.017″/0.43 mm to 0.026″/0.66 mm, and a working length of 65 cm.

The tapering of the tip angle 20 of the flexible tip 15 is designed or constructed with a lesser angle of a range of zero to five degrees to navigate the kink or a bend in the lead catheter. With the lesser angle, the flexible tip 15 can easily be threaded by the medical provider to an entry point in a kink or bend. In embodiments, the flexible tip 15 is configured in a wedge shape that causes the flexible tip 15 to be driven through the kink by the twisting and back/forth motion.

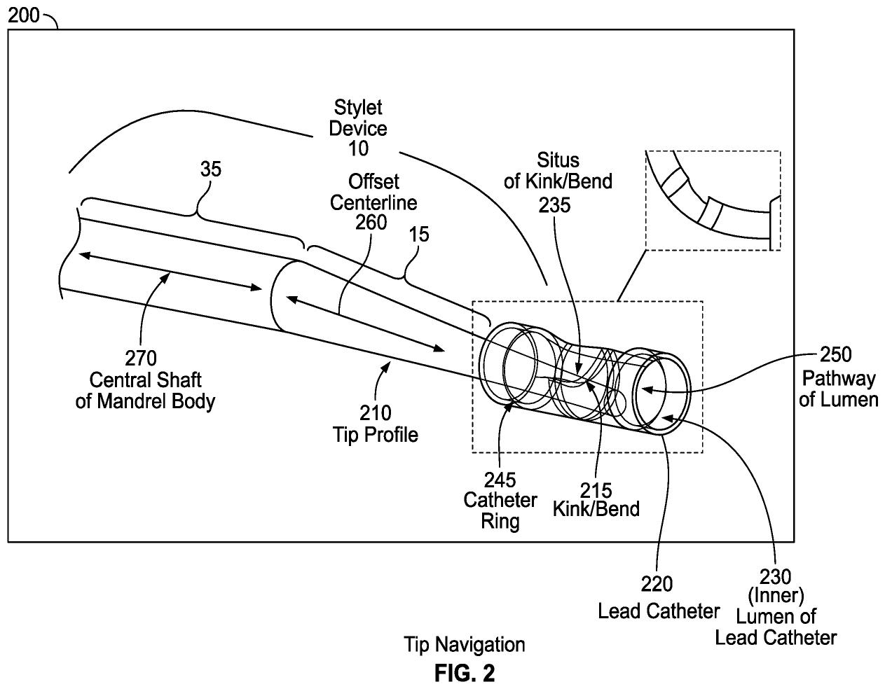

In embodiments, the flexible tip 15 is configured with a central shaft 23 which is offset from a centerline (See FIG. 2 offset of flexible tip to mandrel body) and angled downward towards the distal end 22 of the flexible tip. This translates when the flexible tip 15 is navigated through the lead lumen to be offset from the centerline of the lead lumen and positioned at the distal end to an interior side of the lead lumen to cause the flexible tip 15 to navigate around a kink or a bend in the lead catheter.

In embodiments, the tip diameter increases in a gradual gradient to the full cylindrical portion (of the mandrel body 35) and is also sized according to the LLD clearing stylets. The tip is rounded to reduce the chance of accidental penetration of the lead. Additionally, the rounded shape should be sized according to the coil profile of the leads, thus allowing the tip to maneuver over coils and not become lodged in the coil. A radiopaque band marking 45 is present for navigation, identification within the lead lumen under fluoroscopy.

The tip length 30 is designed to be long enough to allow entry into a sharp bend or kink. The current profile of three (A, D, and L) variables listed here, allows the tip to make entry into a sharp bend in a more flexible manner. The tip would navigate a lot easier around turns, once in the bend or kink, the electrical rotation AND reciprocation would allow the entire clearing (locking) stylet to fully penetrate and clear the path for the LLD. One thing to consider is that we do not want this to be too long to where it consumes too much length and bottoms out at the lead screw not allowing enough LLD to lock. The tip will only clear so much with the current profile, its usage is purely navigational, the full cylindrical-shaped back portion is what clears the path.

The electromechanical device 60 (control handle 67) is configured in a housing 69 that has an ergonomic design and contains buttons and controls to allow the unit to rotate and reciprocate. The rotation speed and reciprocation speed/frequency can be changed and the results view via imaging equipment at the situs of the kink or bend. A marking (not shown) outside of the housing about the marking 45 on the mandrel that is carefully placed about the tip characteristics allows the tracking of tip position when navigating the compromised lumen.

In an embodiment, the LLD used can be either an off-the-shelf LLD or specially configured LLD with enhanced features for the lead extraction procedure. As an example, the SPECTRANETICS® Lead Locking Device (LLD) is an off-the-shelf LLD that may be utilized with the lead navigation system 100 (i.e., lead navigation device) for transvenous removal of implanted pacing or defibrillator leads.

In an embodiment, the electromechanical mechanism 62 of the electromechanical handle (control handle) 60 applies both a rotational movement to twist the stylet device 10 in the kink or bend, and a reciprocal or hammer-drill movement to push the catheter back to an original position or state. The electromechanical mechanism 62 applies a torque to the central shaft 23 of the flexible tip 15, and a translational longitudinal force to the center shaft of the mandrel body 35 that results in both types of forces transmitted to the flexible tip 15 to clear the pathway.

FIG. 2 illustrates a diagram of a lead navigation device operably positioned in a lead catheter at a situs of kink to apply a set of forces to overcome the resistance of kink in accordance with an embodiment. In FIG. 2 there is shown a stylet device 10 with a mandrel body 35 and a flexible tip 15 that is configured with a flexible tip profile where the distal end of the flexible tip is inserted in a lead catheter 220 with catheter rings 245. The flexible tip 15 is positioned at a situs 235 of kink 215 and is shown pressing against the kink 215 to exert a force to overcome the resistance of the kink 215 to flex the lead catheter back to an original position.

In embodiments, the flexible tip 15 presses against a side of the lumen 230 to exert forces caused by reciprocal and rotational motions caused by the electromechanical mechanism 62 to push the side of the lumen 230 back to an original position or in a manner to open the pathway 250 of the lumen to enable an LLD or other instruments used in the lead extraction procedure to pass by the kink 215.

In embodiments, the tip profile 210 configures the flexible tip 15 with an offset centerline 260 (or center shaft) position with a gradual incline that forms a wedge at the distal end 22 of the flexible tip 15. The centerline 260 of the flexible tip 15 is offset from the centerline 270 (or center shaft) of the mandrel body 35 and enable the distal end 22 of the flexible tip 15 to penetrate under the kink 215 and to apply pressure to the kink 215 as it threads forward in a longitudinal direction past the kink 215. That is, the sides of the flexible tip 15 exert pressure against the kink 215 and this pressure continues to expand the pathway 250 up until the mandrel body 35 is reached as the stylet device 10 traverses through the lead catheter 220. Pathway 250 is expanded to the diameter of the lead catheter. Hence, the diameter of the mandrel body 35 is sized to the diameter of the lead catheter 220 to expand the pathway of the lead catheter to the desired width. In embodiments, the diameter is expanded up to the width of the original catheter pathway.

In embodiments, when the pressure exerted against a wall or side of the lumen 230 of the lead catheter 220 is above a threshold of resistance of the wall or side of the lead catheter 220, the lead catheter 220 will flex back to an original position, and the kink 215 or bend will dissipate, and the radius of the lead catheter 220 is no longer restricted.

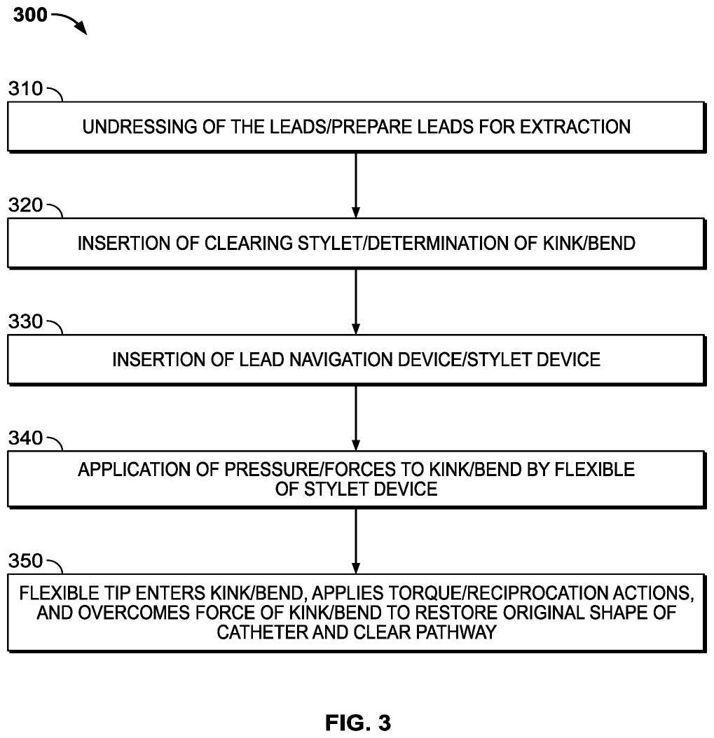

FIG. 3 is a flowchart using the lead navigation system to remove a kink or bend in the lead catheter by the stylet device in accordance with various embodiments.

At step 310 of a lead extraction procedure, a lead extracting medical provider will cut the tip of the leads off to prepare the leads for extraction and for use with a Lead Locking Device (LLD) by the medical provider by undressing each of the leads by removing the insulation and decoupling the outer coil carefully to gain access to the inner coil. At step 320, once the inner coil is exposed, an expanding stylet or mandrel is inserted to expand the inner coil in preparation for insertion of the clearing stylet (of appropriate size), which clears the path of the inner coil for later insertion of the lead locking device. If there is resistance while the medical provider is threading the clearing stylet (i.e., the clearing is blocked, obstructed, or restricted and the clearing stylet cannot be inserted the full length of the lead catheter); the medical provider may determine there is a kink or bend in the lead catheter. In addition, the medical provider may determine a situs of the kink or bend in the lead catheter. As the path must be clear so that that the lead locking device can be inserted far enough into the lead and deployed properly. In this lead preparation stage, the medical provider than at step 330, can elect to insert the lead navigation device (i.e., the stylet device) that is connected to a control handle (electromechanical device 60) and thread the mandrel body 35 with the flexible tip 15 to the situs 225 of the kink or bend (as shown in FIG. 2). At step 340, the medical provider applies pressure via the flexible tip to the kink or bend (i.e., applies force to the lumen wall) to overcome the bend or kink resistance caused by the lead catheter. In embodiments, the mandrel body (of specified diameter) connected to the flexible tip which has a half-tapered tip profile enables the medical provider to traverse the tapered tip around the kink and as snaked down the pathway of the lumen expands the cavity of the catheter to cause the kink to be eliminated. In the case of a kink that is at an extreme angle (e.g., approximately 90 degrees), the long length of the tapered tip can allow the tip to pierce the kink and make entry into the sharp kink. At step 350, once the flexible tip of the stylet device has entered the kink, the motorized portion will rotate and reciprocate (in and out when looking through) to overcome the force of the kink, and eventually bending it back to its original shape and clearing the path allowing correct positioning of lead locking device during deployment.

In embodiments, an apparatus for assisting in navigating an endoscopic instrument is described that includes a stylet device configured to insert a lumen in catheter including the first part of a tip; and the second part of a body for connection to the tip when deployed in the lumen; wherein the tip of the stylet device is configured to: apply pressure at a blockage in the catheter; remove the blockage by the movement of the tip pressing against the blockage in the catheter to overcome the resistance of the blockage; and enable opening by removal of the blockage to thread the endoscopic instrument past during an endoscopic procedure.

Those of skill in the art will appreciate that the various illustrative logical blocks, modules, circuits, and algorithm steps described in connection with the embodiments disclosed herein may be implemented as electronic hardware, computer software, or combinations of both. Some of the embodiments and implementations are described above in terms of functional and/or logical block components (or modules) and various processing steps.

However, it should be appreciated that such block components (or modules) may be realized by any number of hardware, software, and/or firmware components configured to perform the specified functions. To clearly illustrate the interchangeability of hardware, various illustrative components, blocks, modules, and steps have been described above generally in terms of their functionality. Whether such functionality is implemented as hardware or software depends upon the application and design constraints imposed on the overall system.

Skilled artisans may implement the described functionality in varying ways for each application, but such implementation decisions should not be interpreted as causing a departure from the scope of the present invention. In addition, those skilled in the art will appreciate that the embodiments described herein are merely implementations.

In this document, relational terms such as first and second, and the like may be used solely to distinguish one entity or action from another entity or action without necessarily requiring or implying any actual such relationship or order between such entities or actions. Numerical ordinals such as “first,” “second,” “third,” etc. simply denote different singles of a plurality and do not imply any order or sequence unless specifically defined by the claim language. The sequence of the text in any of the claims does not imply that process steps must be performed in a temporal or logical order according to such sequence unless it is specifically defined by the language of the claim. When “or” is used herein, it is logical or mathematical or, also called the “inclusive or.” Accordingly, A or B is true for the three cases: A is true, B is true, and A and B are true. In some cases, the exclusive “or” is constructed with “and;” for example, “one from the set A and B” is true for the two cases: A is true, and B is true.

Furthermore, depending on the context, words such as “connect” or “coupled to” used in describing a relationship between different elements do not imply that a direct physical connection must be made between these elements. For example, two elements may be connected to each other physically, electronically, logically, or in any other manner, through one or more additional elements.

While at least one embodiment has been presented in the foregoing detailed description of the invention, it should be appreciated that a vast number of variations exist. It should also be appreciated that the embodiment or embodiments are only examples and are not intended to limit the scope, applicability, or configuration of the invention in any way. Rather, the foregoing detailed description will provide those skilled in the art with a convenient road map for implementing an embodiment of the invention. It is understood that various changes may be made in the function and arrangement of elements described in an embodiment without departing from the scope of the invention as set forth in the appended claims.

What is claimed is:

1. An apparatus to assist in a lead extraction procedure of a catheter, the apparatus comprising:
a stylet device configured to insert into a lumen of the catheter comprising:
a first part of a tip; and
a second part of a body wherein the body is configured at an end for connection to the tip when deployed in the lumen of the catheter;
wherein the tip of the stylet device is configured to:
apply pressure at an obstruction in the catheter:
remove the obstruction by movement against the obstruction to cause a clearing of obstruction in lumen of the catheter; and
enable opening of the catheter for a lead locking device (LLD) to navigate past during the lead extraction procedure.

2. The apparatus of claim 1, wherein the obstruction in the catheter comprises a kink in the catheter.

3. The apparatus of claim 2, further comprising:
wherein the tip of the stylet device is configured as rounded at the end to prevent damage or perforation to the lumen when pressed against the obstruction by the movement to clear the obstruction.

4. The apparatus of claim 3, further comprising:
wherein the tip is configured in accordance with a flexible tip profile that comprises a tip length, a tip diameter, and a taper angle.

5. The apparatus of claim 4, further comprising:
wherein the tip length of the flexible tip profile is half-tapered to penetrate the kink of the catheter wherein the kink is sharply angled causing closure of the catheter.

6. The apparatus of claim 5, further comprising:
wherein the tip diameter is sized in accordance with a coil profile of the catheter to enable the tip to move inside the catheter and to prevent lodgement in a coil of the catheter.

7. The apparatus of claim 6, further comprising:
wherein the taper angle of the flexible tip profile is a lesser angle of a range of zero to five degrees to navigate the kink or a bend in the catheter wherein at an entry point the tip is configured in a wedge shape to be driven through the kink by the movement.

8. The apparatus of claim 7, further comprising:
wherein the tip is configured with a shaft that is offset from a centerline to navigate through the lumen while positioned at a side of the lumen for the movement around the kink or the bend in the catheter.

9. The apparatus of claim 8, further comprising:
wherein an electromechanical handle mechanism attached to the stylet device comprises at least a rotational motor to rotate the tip to overcome resistance of the kink in the catheter to unkink the catheter.

10. The apparatus of claim 9, further comprising:
a housing of the electromechanical handle mechanism configured for ergonomic use to provide the movement to overcome the resistance of the kink in the catheter.

11. An apparatus for assisting in navigating an endoscopic instrument, the apparatus comprising:
a stylet device configured to insert a lumen in catheter comprising:
a first part of a tip; and
a second part of a body for connection to the tip when deployed in the lumen;
wherein the tip of the stylet device is configured to:
apply pressure at a blockage in the catheter:
remove the blockage by movement of the tip pressing against the blockage in the catheter to overcome resistance of the blockage; and
enable opening by removal of the blockage to thread the endoscopic instrument past during an endoscopic procedure.

12. The apparatus of claim 11, wherein the blockage comprises a kink in the catheter.

13. The apparatus of claim 12, further comprising:
wherein the tip of the stylet device is rounded at an end to press against the blockage without damaging the catheter.

14. The apparatus of claim 13, further comprising:
wherein the tip is configured in accordance with a flexible tip profile that comprises a tip length, a tip diameter, and a taper angle.

15. The apparatus of claim 14, further comprising:
wherein the tip length of the flexible tip profile is half-tapered to enable the tip to penetrate the kink in the catheter.

16. The apparatus of claim 15, further comprising:
wherein the tip diameter of the flexible tip profile is sized in accordance with a coil profile of the catheter to enable the tip to move inside the catheter.

17. The apparatus of claim 16, further comprising:
wherein the taper angle of the flexible tip profile is a lesser angle of a range of zero to five degrees to navigate about the kink in the catheter wherein at an entry point the tip is a wedge shape to be driven through the kink.

18. The apparatus of claim 17, further comprising:
the tip is offset from a centerline and positioned at a side of a lumen in the catheter to move around a kink.

19. The apparatus of claim 18, further comprising:
wherein an electromechanical handle mechanism is coupled to the tip and comprises at least a rotational motor to move the tip in a manner to overcome resistance of the kink.

20. The apparatus of claim 19, further comprising:
a housing of the electromechanical handle mechanism configured for ergonomic use to provide movement to overcome resistance of the kink.
